(19) United States
(12) Patent Application Publication
(10) Pub. No.: US 2026/0325456 A1
(43) Pub. Date: Oct. 22, 2026

(54) METHOD OF REDUCING NITROSAMINE LEVELS IN PHARMACEUTICAL PRODUCTS AND IMPROVED FORMULATIONS THEREOF

(71) Applicant: Sun Pharmaceutical Industries Limited, Mumbai (IN)

(72) Inventors: Joyeeta Dutta CHOUDHURY, Gurugram (IN); Subham Kumar SONI, Gurugram (IN); Vinay KUMAR, Gurugram (IN); Ashish KUMAR, Gurugram (IN); Sumit MADAN, Gurugram (IN); Romi SINGH, Gurugram (IN)

(21) Appl. No.: 19/696,946

(22) Filed: Jun. 3, 2026

Related U.S. Application Data
(63) Continuation of application No. PCT/IB2024/062213, filed on Dec. 4, 2024.

(30) Foreign Application Priority Data
Dec. 5, 2023 (IN) 202321082785

(51) Int. Cl. A61K 9/50; A61J 1/03; A61K 9/48; A61K 31/155

(52) U.S. Cl. CPC A61K 9/5078; A61J 1/035; A61K 9/485; A61K 9/4858; A61K 9/4866; A61K 9/501; A61K 9/5015; A61K 9/5042; A61K 9/5047; A61K 9/5089; A61K 31/155

(57) ABSTRACT

The present invention provides a pharmaceutical formulation comprising an amine-containing pharmaceutically active agent, wherein said composition comprises one or more components selected from a low DMA containing active agent, a nitrite scavenger and/or a pH modifier/buffer. The use of the pH modifier/buffer in the formulation provides a pH in the range of about pH 2 to about pH 10. The disclosure also provides a packaged pharmaceutical formulation comprising an amine-containing pharmaceutically active agent, wherein said packaging i) comprises an oxygen absorbent and/or ii) is breathable or vented to allow diffusion of N-Nitrosamine, such as NDMA, out of the packaging.

### FIELD OF THE INVENTION

The present invention provides an improved pharmaceutical formulation and method of preparing pharmaceutical formulations to ensure that nitrosamine levels remain low, even after extended storage. The improved formulations are provided with characteristically low nitrosamine levels.

### BACKGROUND OF THE INVENTION

N-Nitrosodimethylamine (NDMA) is an organic compound with the formula (CH₃)₂NNO which is formed by the combination of dimethyl amine with nitrites. Whilst NDMA is a known common environmental contaminant, nitrosamine such as NDMA are known to be carcinogenic. Chronic ingestion of NDMA is postulated to cause an increase in liver and other cancers. A Dutch study (Pottegard A, Kristensen K B, Ernst M T, Johansen N B, Quartarolo P, Hallas J. Use of N-nitroso-dimethylamine (NDMA) contaminated valsartan products and risk of cancer: Danish nationwide cohort study. BMJ. 2018; 362) has reported that NDMA-contaminated medicines are associated with a small increase in risk for overall cancer, and the cancer-causing impact of NDMA is understood to be cumulative so that contamination of medicines taken over long periods for chronic conditions are particularly of concern.

Metformin is a highly successful drug used for lowering blood glucose and is considered to be a safe and effective treatment for Type 2 diabetes, and is the third most prescribed drug in India. However, in December 2019, the FDA noted potential NDMA contamination of metformin products, and in particular potential NDMA contamination of extended-release (ER) formulations of metformin. The accepted level of NDMA within metformin products is 0.048 ppm, for a maximum dose of 2 g/day of metformin. The concern associated with NDMA led to a voluntary recall of the formulations to allow further assessment of the NDMA content.

An independent analysis found that 42% of the batches analysed independently exceeded the FDA's daily acceptable intake limit for NDMA (see Ajith Kumar P V, and Deepa M., Metformin and nitrosamine impurities, Int. J. Res. Med. Sci., 2020 October; 8(10): 3778-3784).

It has been postulated that the NDMA could be as a result of the manufacturing process of metformin itself, which involves the use of dimethylamine (DMA), so that the current focus is on examination of the manufacturing process to eliminate contamination by any unreacted DMA within the metformin product (see Ajith Kumar P V, and Deepa M., supra).

The same issue regarding potential NDMA contamination is also understood to be of concern for other amine containing pharmaceutically active agent, such as valsartan and ranitidine.

There is therefore an urgent need for a modified process for formulations (including extended-release formulations) of such active ingredients which maintain low levels of NDMA content (i.e., for example, for metformin within the acceptable level of 0.048 ppm for a maximum dose of 2 g/day of metformin).

The present invention provides methods and formulations which address such needs.

### SUMMARY OF THE INVENTION

The present inventors have surprisingly found that unacceptably high levels of N-Nitrosamines (such as NDMA) within amine-containing pharmaceutically active agents (such as metformin, valsartan, ranitidine and the likes) does not predominantly occur as a result of contamination through manufacturing, but also is due to the presence of nitrate/nitrite groups or nitrosating agents within the pharmaceutical products which allows the formation of N-nitrosamines over time, during storage.

Since removal of the nitrate groups is not an option (since the nitrate groups are required for the activity of the pharmaceutically active agents), the present invention also set out a method of scavenging the nitrite/nitrate groups and thereby preventing the formation of N-Nitrosamines (such as NDMA) within the formulations. Novel formulations are also described.

In one aspect according to the present invention, the composition disclosed herein uses a low dimethyl amine (DMA) containing active, wherein the DMA in the active is present at not more than 300 ppm.

In another aspect according to the present invention, the composition disclosed herein uses excipients having lower quantities of nitrites or nitrates or devoid of these.

In yet another aspect according to the present invention, the composition disclosed herein use a pH modifier or buffering agent to provide a pH less than 10.

In yet another aspect according to the present invention, the composition disclosed herein uses a pH modifier or buffering agent to provide a pH in a range of 2-10, preferably 2-8, more preferably 2-6.

In yet another aspect according to the present invention, the composition disclosed herein uses a nitrite/nitrate scavenger.

In a further aspect according to the present invention, the composition disclosed herein characteristically provides an active ingredient not in solution form.

In some embodiments, the invention comprises a two-part pharmaceutical composition of an amine drug or a pharmaceutically acceptable salt thereof and excipients, wherein the composition comprises:
a first part comprising:
   a drug layered core, wherein the drug layer is coated on an inert core;
   a water insoluble release-controlling polymer coated on the drug layered core; and
   a drug coating over the release control polymer coating, and
a second part comprising a vehicle devoid of drug;
wherein a sulfite salt of alkali metal is present in the first part of the composition in close association with both the drug layered core and in coating over the release control polymer, and
wherein the ratio of the sulfite salt of alkali metal to drug in the composition is present in a ratio of 2.5:80 in drug layered core and 2.5:20 in drug coating.

In some embodiments, the present invention comprises a pharmaceutical formulation comprising metformin as an amine-containing pharmaceutically active agent, and wherein said formulation exhibits levels of NDMA below 0.048 ppm after storage for 7 days or longer.

In some embodiments, the invention discloses a two-part pharmaceutical composition comprised of an amine drug or a pharmaceutically acceptable salt thereof and excipients, wherein the composition comprises:
a first part comprising:
   a drug layered core, wherein the drug layer is coated on an inert core;
   a water insoluble release-controlling polymer coated on the drug layered core; and
   a drug coating over the release control polymer coating, and
a second part comprising a vehicle devoid of drug;
wherein a buffering agent is present in the second part of composition.

In a preferred embodiment, the composition of the present invention maintains NDMA content within the maximum allowable limit of 0.048 ppm after 9 months at 25 °C/60% RH.

In a preferred embodiment, the composition of the present invention maintains NDMA content within the maximum allowable limit of 0.048 ppm after 6 months at Accelerated stability studies.

The aforementioned aspects and embodiments, and other aspects, objects, features and advantages of the present invention will be apparent from the following detailed description.

### DETAILED DESCRIPTION OF THE INVENTION

As used herein the following definitions apply unless clearly indicated otherwise. It should be understood that unless expressly stated to the contrary, the singular forms “a” “an” and “the” include plural reference unless the context clearly dictates otherwise.

By “about” we mean a value, which is plus or minus 10% of the stated value.

By “an amine-containing pharmaceutically active agent” we mean a pharmaceutically active agent (i.e., drug) containing one or more primary, secondary or tertiary amine groups.

By “nitrite scavenger” or “nitrate scavenger” we mean a component in the formulation which reduces or prevents N-nitrosation by scavenging nitrite there by preventing formation of N-nitrosamines.

By “amelioration” we mean that the severity of the disease or disorder is reduced, or that progression of the disease or disorder into a more severe presentation is reduced or delayed.

“Therapeutically effective amount” as used herein refers to the amount of a therapeutic agent that is effective to alleviate the target disease or disorder.

“Patient” as used herein refers to any human or nonhuman animal (e.g., primates, sheep, dogs, cats, horses, cows, chickens, amphibians, reptiles and the like).

“Treatment” as used herein refers to cure the disease and/or disorder as rapidly as possible and the prevention of progression to severe disease.

“Accelerated stability studies” include stability conditions at 40 °C/75% RH.

The present invention is based on the surprising finding that effect of one or more factors selected from:
pH,
low DMA content of active, and
nitrite or nitrate scavenger in formulations of an amine-containing pharmaceutically active agent mitigates against the build-up of undesirable Nitrosamine impurities.

In one aspect of the present invention, the composition disclosed herein does not comprise a drug in a vehicle for reconstitution preparation.

In one aspect, the present invention provides a pharmaceutical formulation comprising an amine-containing pharmaceutically active agent, wherein said composition comprises a nitrite/nitrate scavenger. In a preferred embodiment, the formulation is a suspension pharmaceutical formulation comprising a powder for reconstitution.

In one aspect, the present invention provides a pharmaceutical formulation comprising an amine-containing pharmaceutically active agent, wherein said composition comprises a pH modifier to provide a pH in the range of about 2 to about 10. In a preferred embodiment, the formulation is a suspension pharmaceutical formulation comprising a powder for reconstitution.

In one aspect, the present invention provides a pharmaceutical formulation comprising an amine-containing pharmaceutically active agent, wherein said composition comprises a nitrite/nitrate scavenger and a pH in the range of about 2 to about 10. In a preferred embodiment, the formulation is a suspension pharmaceutical formulation comprising a powder for reconstitution.

In a related aspect of the present invention, the active pharmaceutical agent in the disclosed composition is not in solution form.

The term “amine drug” as used herein the description, means a drug or its salt that has amine group present in the structure as primary, secondary, tertiary, or quaternary amines. The most commonly used amine drugs that can be used according to the present invention, including but not limited to, sartans, antidiabetic drugs, antihistamines, and antibiotics; e.g., the drugs include amoxicillin, bendroflumethiazide, betahistine, bisoprolol, bromazepam, carvedilol, cetirizine, desloratadine, diclofenac, doxylamine, duloxetine, enalapril, ergometrine, felodipine, flecainide, fluoxetine, hydrochlorothiazide (HCT), levofloxacin, metoprolol, metformin, mirabegron, mirtazapine, moxifloxacin, nebivolol, opipramol, pergolide, propranolol, quetiapine, ramipril, roxithromycin, sertraline, sotalol, sumatriptan, sitagliptin, valsartan, varenicline, ranitidine, gliclazide, prucalopride, clarithromycin, oxcarbazepine, sunitinib, rivaroxaban, citalopram, escitalopram, tamsulosin, diltiazem, amlodipine or pharmaceutically acceptable salt thereof, and combination thereof.

In one aspect, the amine-containing pharmaceutically active agent is selected from metformin, valsartan, losartan and/or ranitidine.

In a preferred embodiment, the amine-containing pharmaceutically active agent is metformin.

In an embodiment according to the present invention, a nitrite/nitrate scavenger is present in the composition in an amount not more than 50% w.r.t. amine containing pharmaceutically active agent.

In some embodiments of the present invention, the pharmaceutical formulation contains a nitrite/nitrate scavenger. The nitrite/nitrate scavenger (for example, ascorbic acid, propyl gallate, sodium metabisuphite, Vitamin E, butylated hydroxyanisole (BHA), and butylated hydroxytoluene (BHT)) can be present within the formulation in an amount from about 0.01% w/w to about 50% w/w with respect to amine containing pharmaceutically active agent.

In an embodiment, the nitrite/nitrate scavenger is present within the formulation in an amount from about 0.01% w/w to about 30% w/w with respect to amine containing pharmaceutical active agent.

In another embodiment, the nitrite/nitrate scavenger is present within the formulation in an amount from about 0.01% w/w to about 10% w/w with respect to amine containing pharmaceutical active agent.

In some embodiments, the pharmaceutical formulation according to the present invention is without any nitrite/nitrate scavenger, wherein such formulation is characterized by a pH in the range of pH 2 to pH 10, preferably pH 2 to pH 8, more preferably pH 2 to pH 6, and the formulation comprises the active ingredient with a lower content of dimethyl amine (DMA), preferably no more than (“NMT”) 100 ppm, more preferably NMT 10 ppm.

In one embodiment, the present invention provides compositions with reduced NDMA impurity. In one aspect, such composition is devoid of any nitrite/nitrate scavenger. In one aspect, the composition reduces NDMA by use of an active ingredient with low DMA content. In another aspect, the composition reduces NDMA by use of a pH modifier to provide a pH of the formulation in the range of 2-10. In yet another aspect, the active in the formulation is in solid form. In a further aspect, the active in the formulation is not in solution form during storage.

In some embodiments, the pharmaceutical formulation contains a pH modifier in the formulation. Generally, the pH modifier will be used in an amount suitable to provide a pH less than 10.

In some embodiments, the pharmaceutical formulation contains a pH modifier in the formulation. Generally, the pH modifier will be used in an amount suitable to provide a pH between pH 2 and pH 10. Exemplary pH modifiers are any excipient which can raise the pH above 2, including, but not limited to, citric acid monohydrate, trisodium citrate dihydrate, meglumine, magnesium oxide and/or sodium hydroxide, polyethylene oxide.

In some embodiments of the present invention, the pharmaceutical formulation comprises a buffering agent. Generally, the buffering agent will be used in an amount suitable to provide a pH between pH 2 and 10. Exemplary buffering agent are ones which can maintain the pH between about pH 2 to about pH 8. In some aspects of the above embodiment, the pH of the buffer ranges between pH 2 to about pH 6. Some of the exemplary buffers that can be used in the formulation are citrate buffer, phosphate buffer, acetate buffer, and the like.

In a liquid composition, a buffering agent may be added to maintain the pH between 2-10, preferably pH between 2-8, more preferably pH between 2-6.

Optionally, the pharmaceutical formulation of the present invention is an extended-release formulation that releases the amine-containing pharmaceutically active agent over an extended period of time. For example, the extended-release formulation can provide release of at least one active ingredient over an extended period of time, for example, over at least 0.5 hours, at least 2 hours, at least 4 hours, at least 5 hours, at least 6 hours, or at least 8 hours.

In a further aspect, the present invention provides the use of a pharmaceutical formulation as described above, wherein said amine-containing pharmaceutically active agent is metformin, and the formulation is for use in the treatment of diabetes, for example, type II diabetes.

In a further aspect, the present invention provides a method of treatment of diabetes, wherein the composition as described above containing metformin as said amine-containing pharmaceutically active agent is administered to a patient.

In another aspect, the present invention provides a method of treatment of diabetes, wherein the composition as described above containing metformin as said amine-containing pharmaceutically active agent is administered to a patient in combination with other amine containing pharmaceutically active agents.

The FDA has stipulated an “Allowable Limit” of NDMA of 96 ng daily (see, for example, “Control of Nitrosamine Impurities in Human Drugs, Guidance for Industry”, published by the FDA, Revision 1, February 2021 and available at https://www.fda.gov/regulatory-information/search-fda-guidance-documents/control-nitrosamine-impurities-human-drugs). The allowable amount of NDMA for a pharmaceutically active agent can be calculated in ppm as 96 ng/Maximum Daily Dosage (MDD) of the specific pharmaceutically active agent in mg. So, for example: metformin has an MDD of 2000 mg, so the maximum ppm allowable is 96/2000=0.048 ppm.

Thus, where the pharmaceutical formulation of the present invention comprises metformin as an amine-containing pharmaceutically active agent, the formulation desirably exhibits levels of NDMA below 0.048 ppm. Desirably, such a formulation exhibits levels of NDMA below 0.048 ppm after storage for at least 7 days or longer (for example, 14 days or longer) during stability. In one embodiment, the formulation according to present invention is stable after reconstitution for at least 7 days. In one embodiment, the formulation is packaged in a container closure system of a round bottle pack. Additionally, the present invention provides the use of such a pharmaceutical formulation for use in the treatment of diabetes, for example, type II diabetes.

In some embodiments according to the present invention, the molar ratio of drug to the nitrite/nitrate scavenger plays an important role in reducing the nitrosamine formation in the drug product. In one aspect, such use of drug and nitrite/nitrate scavenger in specific molar ratios plays role in controlling nitrosamine formation.

In a further aspect, the present invention provides a packaged pharmaceutical formulation comprising an amine-containing pharmaceutically active agent, wherein i) said packaging comprises an oxygen absorbent and/or ii) said packaging is breathable or vented to allow diffusion of N-Nitrosamine out of the packaging.

In yet another aspect, the packaging comprises an oxygen absorbent, for example, an oxygen absorbent liner or additive. The oxygen absorbent prevents or reduces the amount of N-Nitrosamine formed during storage. The N-Nitrosamine may be NDMA.

In one embodiment, the packaging according to present invention can be breathable or vented to remove N-Nitrosamine formed during storage. The N-Nitrosamine may be NDMA. Optionally, the packaging can be a vented blister pack. Optionally, the pack can include a breathable liner that can expel the NDMA outside of the pack. An example can be a molecular sieve with a breathable liner.

In a further aspect, the present invention provides a method of storing a pharmaceutical formulation comprising an amine-containing pharmaceutically active agent, wherein said formulation is packaged into i) packaging which comprises an oxygen absorbent and/or ii) packaging which is sufficiently breathable or vented to cause diffusion of N-Nitrosamine formed during storage out of the packaging. The N-Nitrosamine may be NDMA. Optionally, the levels of NDMA within the amine-containing pharmaceutically active agent are maintained below the FDA stipulated level for the specific active agent for the period of storage. In one aspect, the suspension is stored during in-use period at 25 °C for at least 7 days.

Optionally, the method includes the step of adding a buffer to provide a pH in the range of pH 2-pH 10 to the formulation, preferably pH 2-8, more preferably pH 3-6.

Optionally, the formulation according to the present invention can conveniently contain other excipients and carriers.

In some embodiments, the formulation is in the form of an oral suspension, for example, is formulated as a suspension. Optionally, the formulation is an extended-release suspension formulation that provides a slow release of at least one active ingredient over an extended period of time, for example, at least 0.5 hours, at least 2 hours, at least 4 hours, at least 5 hours, at least 6 hours, or at least 8 hours.

In some embodiments, the formulation is in the form of granules or pellets or a powder for tablet preparation, for example, is formulated as a coated tablet, or powder or pellet for filling into capsule. Optionally, the formulation is an extended-release formulation, that is provides a slow release of at least one active ingredient over an extended period of time, for example, at least 0.5 hours, at least 2 hours, at least 4 hours, at least 5 hours, at least 6 hours, or at least 8 hours.

In some embodiments, the formulation is in the form of a ready to use formulation. Optionally, the formulation is an extended-release formulation that provides a slow release of at least one active ingredient over an extended period of time, for example, at least 0.5 hours, at least 2 hours, at least 4 hours, at least 5 hours, at least 6 hours, or at least 8 hours.

In some embodiments, the formulation according to present invention can have one additional drug other than metformin for administration to the patient in need thereof. In one aspect, the composition has controlled levels of nitrosamine impurity throughout the shelf life.

In some embodiment, the formulation according to the present invention can be a combination composition of two or more amine-containing drugs, wherein the composition is characterized by controlled levels of nitrosamine impurity in the composition. In one aspect, the composition comprises low dimethyl amine containing active ingredients and the pH of the composition is in range of pH 2-10.

In one embodiment, the formulation according to the present invention is a combination composition of metformin and sitagliptin, wherein the composition is characterized by nitrosamine impurity below 0.048 ppm in the composition.

In one aspect of the above embodiment, the combination composition comprises carbon or charcoal in the packaging of the combination composition.

In one embodiment, the formulation according to the present invention is a combination composition of metformin with two or more drugs, wherein the composition is characterized by controlled levels of nitrosamine impurity.

In one aspect of the above embodiment the additional drug(s) is selected from alogliptin, canagliflozin, dapagliflozin, empagliflozin, ertugliflozin, glipizide, glyburide, linagliptin, saxagliptin, sitagliptin, pioglitazone, rosiglitazone, and pharmaceutically acceptable salts thereof.

In one aspect of the above embodiment, the combination composition comprises a carbon or charcoal in the packaging of the combination composition.

In one embodiment, the formulation according to the present invention is prepared by a process comprising:
a) Dissolving a drug substance, binder and nitrite/nitrate scavenger in an aqueous solvent to get a drug dispersion and coating the dispersion on an inert core and drying the formed pellet;
b) Dissolving a release-controlling polymer and plasticizer in a ketonic aqueous solvent to get a uniform extended-release coating dispersion and coating the drug coated core pellets with the dispersion and drying the extended-release (“ER”) coated pellet;
c) Dissolving a drug substance, binder and nitrite/nitrate scavenger in an aqueous solvent to get a uniform dispersion and coating the dispersion on the ER coated pellets and drying;
d) Dissolving a film forming agent in an aqueous solvent, and dispersing an anti-tacking agent in the aqueous solution and coating the drug coated ER pellets and drying, followed by lubrication with a lubricant to get component 1; and
e) Separately dispersing preservatives, osmogen, one or more suspending agents, buffers, sweeteners, flavors in aqueous solvent with continuous stirring to get a vehicle as component 2.
f) Packing both component 1 and component 2 as per suitable packing practice.

Exemplary embodiments of the disclosed invention:
a two-part pharmaceutical composition of an amine drug or a pharmaceutically acceptable salt thereof and excipients, wherein the composition comprises:
a first part comprising:
a) a drug layered core, wherein the drug layer is coated on an inert core;
b) a water insoluble release-controlling polymer coated on the drug layered core; and
c) a drug coating over the release control polymer coating, and
d) a second part comprising a vehicle devoid of drug;
wherein a sulfite salt of alkali metal is present in the first part of composition in close association with both the drug layered core and in the drug coating over the release control polymer, and
wherein the ratio of the drug to sulfite salt of alkali metal in the composition is present in a ratio of 1:0 to 1:0.5 in drug-layered core and 1:0 to 1:0.5 in drug coating.

In one aspect of the above two-part pharmaceutical composition, the amine drug is metformin.

In another aspect of the above two-part pharmaceutical composition, the release-controlling polymer or agent is selected from a pH dependent polymer and a pH independent polymer, or combination thereof. In one aspect, such release-controlling polymer is water insoluble.

In another aspect of the above two-part pharmaceutical composition, the release-controlling polymer is a pH dependent polymer or agent selected from acrylic copolymers, such as, methacrylic acid and methyl methacrylate copolymers, e.g., Eudragit® L 100 and Eudragit® S 100, methacrylic acid and ethyl acrylate copolymers, e.g., Eudragit® L 100-55 and Eudragit® L 30 D-55, dimethylaminoethyl methacrylate and butyl methacrylate and methyl methacrylate copolymers, e.g., Eudragit® E 100, Eudragit® E PO, methyl acrylate and methacrylic acid and octyl acrylate copolymers, styrene and acrylic acid copolymers, butyl acrylate and styrene and acrylic acid copolymers, and ethylacrylate-methacrylic acid copolymer; cellulose acetate phthalate; cellulose acetate succinates; hydroxyalkyl cellulose phthalates such as hydroxypropylmethyl cellulose phthalate; hydroxyalkyl cellulose acetate succinates such as hydroxypropylmethyl cellulose acetate succinate; vinyl acetate phthalates; vinyl acetate succinate; cellulose acetate trimelliate; polyvinyl derivatives such as polyvinyl acetate phthalate, polyvinyl alcohol phthalate, polyvinyl butylate phthalate, and polyvinyl acetoacetal phthalate; zein; shellac; and mixtures thereof.

In another aspect of the above two-part pharmaceutical composition, the release-controlling polymer is a pH independent polymer selected from cellulosic polymers such as ethyl cellulose, methyl cellulose, hydroxyethyl cellulose, hydroxypropyl cellulose, hydroxyethylmethyl cellulose, hydroxypropylmethyl cellulose, and carboxy methyl cellulose; acrylic copolymers such as methacrylic acid copolymers, e.g., Eudragit® RS, Eudragit® RL, Eudragit® NE 30 D; cellulose acetate; polyethylene derivatives, e.g., polyethylene glycol and polyethylene oxide; polyvinyl alcohol; polyvinyl acetate; gums e.g., guar gum, locust bean gum, tragacanth, carrageenan, alginic acid, gum acacia, gum arabic, gellan gum, and xanthan gum; triglycerides; waxes, e.g., Compritol®, Lubritab®, and Gelucires®; lipids; fatty acids or their salts/derivatives; a mixture of polyvinyl acetate and polyvinyl pyrrolidone, e.g., Kollidon® SR; and mixtures thereof.

In yet another aspect of the above two-part pharmaceutical composition, the vehicle comprises a combination of osmogen and a suspending agent.

In one aspect of the above two-part pharmaceutical composition, the vehicle comprises one or more suspending agents in the composition.

In some aspects of the above two-part pharmaceutical composition, the sulfite salt of an alkali metal is selected from sodium sulfite, sodium bisulfite, sodium metabisulfite, potassium sulfite, potassium bisulfite, potassium metabisulfite, calcium sulfite, calcium bisulfite, calcium metabisulfite or combination thereof.

In yet another aspect of the above said two-part pharmaceutical composition, the amine drug or pharmaceutically acceptable salt thereof is metformin and the dimethyl amine content is not more than 300 ppm, preferably below 290 ppm, 280 ppm, 270 ppm, 260 ppm, 250 ppm, 240 ppm, 230 ppm, 220 ppm, 210 ppm, 200 ppm, 190 ppm, 180 ppm, 170 ppm, 160 ppm, 150 ppm, 140 ppm, 130 ppm, 120 ppm, 110 ppm, 100 ppm, 90 ppm, 80 ppm, 70 ppm, 60 ppm, 50 ppm, 40 ppm, 30 ppm, 20 ppm, 10 ppm, 9 ppm, 8 ppm, 7 ppm, 6 ppm, 5 ppm, 4 ppm, 3 ppm, 2 ppm, or below 1 ppm.

In a further related aspect of the above two-part pharmaceutical composition, the dimethyl amine content of the amine drug or pharmaceutically acceptable salt thereof is not more than 100 ppm, preferably below 90 ppm, 80 ppm, 70 ppm, 60 ppm, 50 ppm, 40 ppm, 30 ppm, 20 ppm, 10 ppm, 9 ppm, 8 ppm, 7 ppm, 6 ppm, 5 ppm, 4 ppm, 3 ppm, 2 ppm, or below 1 ppm.

In yet another aspect of the above two-part pharmaceutical composition, the first part of the composition is a stable extended-release reconstitution powder for suspension composition comprising multiple coated cores of the active amine drug.

In some aspects of the above two-part pharmaceutical composition, the composition is characterized by having a NDMA impurity below 0.048 ppm.

In another embodiment, the pharmaceutical formulation has levels of NDMA of below 96 ng per Maximum Daily Dosage.

In one embodiment according to present invention, the disclosure provides a pharmaceutical formulation comprising metformin as an amine-containing pharmaceutically active agent, and wherein said formulation exhibits levels of NDMA below 0.048 ppm after storage for at least 7 days or longer during in use period.

In yet another preferred embodiment according to the present invention, the disclosure provides a process for preparation of a stable pharmaceutical composition of metformin, which is characterised by a NDMA content below 0.048 ppm, wherein the composition is prepared by a process comprising:
a) mixing a first fraction of low dimethyl amine active amine drug with a sulfite salt of an alkali metal and pharmaceutically acceptable excipients and coating the drug mixture on an inert core;
b) coating the core with a release-controlling polymer to obtain a coated drug core;
c) mixing a second fraction of a low dimethyl amine active drug with a sulfite salt of an alkali metal and coating said mixture on the coated drug core;
d) coating the step c) resulting coated core with a top coating followed by lubrication;
e) separately preparing an aqueous vehicle by mixing osmogen in one or more suspending agents, adding a buffering agent, a preservative and one or more pharmaceutically acceptable excipients to the mixture; and
f) packing separately lubricated coated cores and vehicle.

In one embodiment according to the present invention, the disclosure provides a packaged pharmaceutical formulation comprising an amine-containing pharmaceutically active agent, wherein said packaging comprises an oxygen absorbent and/or is breathable or vented to allow diffusion of N-Nitrosamine out of the packaging.

In one aspect of the present invention, the packaged pharmaceutical formulation has a packaging, i.e., breathable or vented, to remove N-Nitrosamine formed during storage.

In another aspect of the present invention, the packaged pharmaceutical formulation has a packaging that is a vented blister pack.

In yet another aspect of the above embodiment, the packaged pharmaceutical formulation has a packaging which is a pill bottle containing an oxygen absorbent.

In one of the aspects of the above embodiment, the packaged pharmaceutical formulation has silica as oxygen absorbent.

In another aspect of the above embodiment, the packaged pharmaceutical formulation has a dual chamber packaging.

In yet another aspect of the above embodiment, the N-Nitrosamine is NDMA.

In one aspect of the above embodiments, the amine-containing pharmaceutically active agent is metformin and the levels of NDMA present after at least 3 months of storage at accelerated stability condition is 0.048 ppm or less.

In one embodiment, the present invention discloses a pharmaceutical composition of an amine drug or pharmaceutically acceptable salt thereof and excipients, wherein the composition comprises:
a) a drug layered core, wherein the drug layer is coated on an inert core;
b) a water insoluble release-controlling polymer coated on the drug layered core;
c) a drug coating over the release control polymer coating, and
d) a vehicle devoid of drug;
wherein a sulfite salt of alkali metal is present in the composition in close association with the drug layered core and in drug coating over the release control polymer.

In another aspect of the above embodiment, the ratio of the drug to sulfite salt of alkali metal in the composition is present in a ratio of 1:0 to 1:0.5 in drug layered core and 1:0 to 1:0.5 in the drug coating.

In another aspect of the pharmaceutical formulation, the active agent is metformin and the formulation is for use in the treatment of diabetes.

The present invention is illustrated below by reference to the following examples. However, one skilled in the art will appreciate that specific methods and results discussed are merely illustrative of the invention, as innumerable variations, modifications, applications, and extensions of these embodiments and principles can be made without departing from the spirit and scope of the invention.

### EXAMPLES

### Example 1: Various Strategies were Evaluated to Develop a Stable Formulation with Controlled Level of NDMA Impurities

### Example 1 A: Effect of DMA Content of API in Finished Product

| | NDMA (μg/g) | | |
|---|---|---|---|
| | | 40 °C/75% | 40 °C/75% |
| Sample Detail | Initial | RH 1M | RH 3M |
| Drug Coated ER pellets with | 0.0279 | 0.0889 | 0.0900 |
| high DMA content API | | | |
| [(470 ppm) + Vehicle] | | | |
| Drug Coated ER pellets with | 0.0191 | 0.0493 | 0.0745 |
| high DMA content API | | | |
| [(74 ppm) + Vehicle] | | | |

Observation: API with lower level of Dimethylamine (DMA) contents helped in the control of NDMA formation in a drug product.

### Example 1 B: Effect of Change in Excipient from an IR Vehicle Containing Drug

| Sample Detail | NDMA (μg/g) | |
|---|---|---|
| (DMA content of API 470) | Initial | 60 °C/15 Day |
| IR Vehicle with Microcrystalline cellulose and | 0.0199 | 0.6616 |
| carboxy methyl cellulose sodium (20 mg) | | |
| IR vehicle with Sodium CMC (3 mg) | — | 0.0203 |

Observation: Replacement of microcrystalline cellulose and carboxy methyl cellulose sodium with sodium CMC helped in the control of NDMA formation.

### Example 1 C: Effect of Buffer System on NDMA Formation

| | NDMA (μg/g) | |
|---|---|---|
| | | 60 °C/ |
| Sample Detail | Initial | 15 Day |
| Metformin HCL API (100 mg) + Water (465.5 mg) | — | 0.0432 |
| [pH 4-5 with Citrate buffer] | | |
| Metformin HCL API (100 mg) + Water (465.5 mg) | — | 0.0784 |
| [pH 5-6 with Citrate buffer] | | |
| Metformin HCL API (100 mg) + Water (465.5 mg) | — | 0.1202 |
| [pH 6-7 with Citrate phosphate buffer] | | |
| Metformin HCL API (100 mg) + Water (465.5 mg) | — | 0.1059 |
| [pH 7-8 with Phosphate buffer] | | |

Observation: Buffer system with pH 4-5, was better when compared to higher pH buffers in controlling NDMA formation.

### Example 1 D: Effect of Sodium Metabisulphite on NDMA Formation in Finished Product

| | NDMA (μg/g) | | | |
|---|---|---|---|---|
| Sample Detail | | 40 °C/75% | 40 °C/75% | 40 °C/75% |
| (Using Low DMA API) | Initial | RH 1M | RH 3M | RH 6M |
| Batch without Sodium Metabisulphite | ND | 0.0057 | 0.0197 | 0.0775 |
| Batch with 10 mg of Sodium | ND | ND | ND | 0.0041 |
| Metabisulphite | | | | (5M 10 days) |

Observation: Formulation prepared by addition of sodium metabisulfite helped in the control of NDMA formation in a drug product.

Based on above trial experiments as set forth in Examples 1 A-D, formulations were developed which comprised a combination of impurity reduction strategies with controlled level of nitrosamine when compared to existing or available metformin compositions. Details of the formulations are provided below.

### Example 2: General Formulation

| | |
|---|---|
| | % w/w (of total |
| Ingredients | pellet wt.) Range |

Metformin Hydrochloride Top Coated Pellets

| Drug Layering | |
|---|---|
| Inert Core | 18-24 |
| Drug Substance | 25-33 |
| Binder | 2.8-7.2 |
| Nitrite Scavenger | 0.2-2.2 |
| Solvent 1 (Aq. solvent) | Q.S. |
| Extended Release Coating | |
| Release-controlling Polymer | 22-35 |
| Plasticizer | 0.1-2.5 |
| Solvent 2 (Ketonic solvent) | Q.S. |
| Solvent 1 (Aq. Solvent) | Q.S. |
| Drug Coating | |
| Drug Substance | 5.8-9.2 |
| Binder | 0.8-2.8 |
| Nitrite scavenger | 0.2-2.5 |
| Solvent 1 (Aq. Solvent) | Q.S. |
| Top Coating | |
| Film forming agent | 4.7-7.2 |
| Anti-tacking agent | 0.15-2.3 |
| Solvent 1 (Aq. Solvent) | Q.S. |
| Lubrication | |
| Lubricant | 0.2-2.7 |
| Total Pellet wt. (in mg) | 252-308 |
| | % w/w (of total |
| | vehicle wt.) |
| Vehicle | |
| Osmogen | 30-65 |
| Suspending Agent 1 | 2.0 |
| Suspending Agent 2 | 0.14-2.8 |
| Suspending Agent 3 | 0.05-2.2 |
| Suspending Agent 4 | 0.1-2.5 |
| Preservative 1 | 0.1-1.5 |
| Preservative 2 | 0.01-0.5 |
| Flavor | 0.1-2.2 |
| Sweetener | 0.01-0.3 |
| Buffering Agent | 0.1-2.2 |
| Buffering Agent | 0.01-1.2 |
| Base for vehicle | 28-61 |
| Total wt. of vehicle (in mg) | 820-1003 |

### Manufacturing Method:

### Drug Layering:

Dissolved the drug substance, binder and nitrite scavenger in an aqueous solvent to get a drug dispersion, and coated the dispersion on an inert core and then dried the formed pellet;

### Extended Release Coating:

Dissolved a release-controlling polymer and a plasticizer in a ketonic aqueous solvent to get a uniform extended-release coating dispersion and then coated the drug coated core pellets with the dispersion and dried the ER coated pellet;

### Drug Coating:

Dissolved the drug substance, binder and nitrite scavenger in an aqueous solvent to get a uniform dispersion, and coated the dispersion on the ER coated pellets, and dried the pellets;

### Top Coating & Lubrication:

Dissolved a film forming agent in an aqueous solvent, and dispersed an anti-tacking agent in the aqueous solution and then coated the drug coated ER pellets and dried the pellets, followed by lubrication with a lubricant to get component 1; and

### Vehicle:

Dispersed preservatives, osmogen, one or more suspending agents, buffers, sweeteners, and flavors in an aqueous solvent with continuous stirring to get a vehicle as component 2.

Packing both component 1 and component 2 as per suitable packing practice.

### Example 3: Various Exemplary Compositions

| | Quantity (% w/w) | | | | | |
|---|---|---|---|---|---|---|
| Ingredients | A | B | C | D | E | F |
| Drug Layering | | | | | | |
| Microcrystalline Cellulose | 16.38 | 17.84 | 15.70 | 19.95 | 21.25 | 22.97 |
| Metformin HCl | 29.55 | 31.85 | 28.04 | 28.49 | 25.30 | 27.34 |
| Hypromellose | 9.40 | 12.74 | 2.80 | 5.70 | 5.06 | 1.37 |
| Sodium metabisulfite | 0.00 | 0.00 | 14.02 | 0.89 | 1.58 | 0.43 |
| Purified Water | Q.S. | Q.S. | Q.S. | Q.S. | Q.S. | Q.S. |
| Extended Release Coating | | | | | | |
| Ethyl Cellulose | 30.60 | 24.49 | 21.56 | 27.39 | 29.18 | 31.54 |
| Dibutyl Sebacate | 0.80 | 0.60 | 0.53 | 0.68 | 0.72 | 0.78 |
| Acetone | Q.S. | Q.S. | Q.S. | Q.S. | Q.S. | Q.S. |
| Purified Water | Q.S. | Q.S. | Q.S. | Q.S. | Q.S. | Q.S. |
| Drug Coating | | | | | | |
| Metformin hydrochloride | 7.65 | 7.96 | 7.01 | 7.12 | 6.33 | 6.84 |
| Hypromellose | 2.29 | 3.18 | 0.70 | 1.42 | 1.27 | 0.34 |
| Sodium metabisulfite | 0.00 | 0.00 | 3.50 | 0.89 | 1.58 | 0.43 |
| Purified Water | Q.S. | Q.S. | Q.S. | Q.S. | Q.S. | Q.S. |
| Top Coating | | | | | | |
| Hypromellose | 4.13 | 0.00 | 4.59 | 5.83 | 6.21 | 6.71 |
| Magnesium stearate | 0.00 | 0.00 | 0.77 | 0.65 | 0.46 | 0.50 |
| Purified water | Q.S. | Q.S. | Q.S. | Q.S. | Q.S. | Q.S. |
| Lubrication | | | | | | |
| Magnesium stearate | 1.20 | 1.33 | 0.78 | 0.99 | 1.06 | 0.76 |
| Total Pellet wt. (in mg) | 100.00 | 100.00 | 100.00 | 100.00 | 100.00 | 100.00 |
| Vehicle | | | | | | |
| Xylitol | 46.12 | 44.80 | 51.97 | 49.34 | 44.74 | 49.19 |
| Carboxy methyl cellulose | 0.29 | 0.15 | 0.43 | 0.33 | 0.30 | 0.44 |
| sodium | | | | | | |
| Xanthan gum | 0.21 | 0.37 | 0.04 | 0.16 | 0.37 | 0.10 |
| Colloidal silicon dioxide | 0.22 | 0.52 | 0.30 | 0.38 | 0.52 | 0.38 |
| Methyl Paraben | 0.29 | 0.27 | 0.16 | 0.20 | 0.27 | 0.20 |
| Propyl Paraben | 0.02 | 0.03 | 0.02 | 0.02 | 0.03 | 0.02 |
| N&A Strawberry | 0.21 | 0.07 | 0.22 | 0.16 | 0.07 | 0.27 |
| Sucralose | 0.04 | 0.03 | 0.07 | 0.05 | 0.03 | 0.09 |
| Citric Acid monohydrate | 0.42 | 0.51 | 0.47 | 0.48 | 0.51 | 0.59 |
| Trisodium citrate | 0.06 | 0.05 | 0.05 | 0.05 | 0.05 | 0.06 |
| Purified water | 52.12 | 53.18 | 46.27 | 48.81 | 53.10 | 48.66 |
| Total wt. of vehicle (in mg) | 100.00 | 100.00 | 100.00 | 100.00 | 100.00 | 100.00 |

### Example 4: Comparative Stability Analysis of Samples from Example 3

NDMA (μg/g)

| Sample No. | Initial | 3M ACC |
|---|---|---|
| Sample A1 | 0.0730 | — |
| Sample A2 | 0.1445 | — |
| Sample A3 | 0.0719 | — |
| Sample A4 | BLQ | 0.0091 |

### Sample A1-A3 Preparation:

Samples were prepared with high dimethyl amine metformin, without nitrite scavenger in the drug core and without drug coating. The vehicle of the samples was prepared with the drug in the vehicle along with microcrystalline cellulose and carboxy methyl cellulose sodium.

### Sample A4 Preparation:

Sample was prepared with low dimethyl amine metformin (below 10 ppm DMA content), with a nitrite scavenger in the drug core and drug coating. The vehicle of the samples was devoid of drug, microcrystalline cellulose and carboxy methyl cellulose sodium.

### Observation:

Samples of batch A1-A3 were found to have higher nitrosamine content in the initial sample, whereas samples of the A4 batch had controlled levels of nitrosamine.

### Example 5: PH Study of the Sample Formulation at Different pH

Preparation: Samples F1, F2 and F3 were prepared with low dimethyl amine metformin (below 10 ppm DMA content), with a nitrite scavenger in the drug core and drug coating. The vehicle of the samples was devoid of drug, microcrystalline cellulose and carboxy methyl cellulose sodium. The buffer was used to provide different pH conditions in the formulation as provided in the below table:

| Sample Code | pH | NDMA (μg/g) |
|---|---|---|
| F1 | 6.07 | 0.0112 |
| F2 | 10.10 | ND |
| F3 | 2.12 | ND |

Observation: Sample formulations were found to be stable with controlled levels of NDMA in the pH range of 2-10.

Stability study: Stability study was undertaken for composition of Example 3.D. The stability products were placed into an inverted (or horizontal) position and an upright (or vertical) position at 25 °C/60% RH. In some cases, content of NDMA was detected by an analytical system (e.g., gas chromatography/mass spectrometry (GC-MS). Stability Data with respect to content of NDMA are depicted in the table below.

| | | CONTENT OF | | | | |
|---|---|---|---|---|---|---|
| | | N-NITROSODIMETHYLAMINE | | | | |
| | Stability | (NDMA) (ppm) by GC/MS | | | | |
| Batch | condition | 0 | 3 months | 6 months | 9 months | 12 months |
| 1 | 25 +/− 2 °C/ | Not | 0.025 | Below LOD | Below LOQ | NA |
| | 60 +/− 5% RH, | detected | | (Limit of | (Limit of | |
| | Upright, | | | detection) | Quantification) | |
| | 25 +/− 2 °C/ | Not | 0.012 | Below LOD | Below LOQ | NA |
| | 60 +/− 5% RH, | detected | | | | |
| | Horizontal, | | | | | |
| 2 | 25 +/− 2 °C/ | Not | Below LOQ | Not | Below LOQ | NA |
| | 60 +/− 5% RH, | detected | | detected | | |
| | Upright, | | | | | |
| | 25 +/− 2 °C/ | Not | 0.015 | Not | Below LOQ | NA |
| | 60 +/− 5% RH, | detected | | detected | | |
| | Horizontal, | | | | | |
| 3 | 25 +/− 2 °C/ | Not | Below LOD | Below LOD | Below LOQ | Not |
| | 60 +/− 5% RH, | detected | | | | detected |
| | Upright, | | | | | |
| | 25 +/− 2 °C/ | Not | Below LOQ | Below LOD | Below LOD | Not |
| | 60 +/− 5% RH, | detected | | | | detected |
| | Horizontal, | | | | | |

Further stability study of batches was carried out for 25 °C/60% RH at 9 months. Dissolution, impurity and pH data are provided in the table below. The stability products were placed into an inverted (or horizontal) position and an upright (or vertical) position at 25 °C/60% RH.

| Parameter | Specification | Batch 1 | Batch 2 | Batch 3 |
|---|---|---|---|---|
| Dissolution (Mean) | % Release in 5 | 88 | 89 | 90 |
| | hr = NLT 75% | 88 | 88 | 88 |
| Total Degradation | NMT 0.75% w/w | 0.10 | 0.12 | Below |
| product | | | | disregard |
| | | | | limit |
| | | 0.10 | 0.12 | Below |
| | | | | disregard |
| | | | | limit |
| pH | NMT 8.0 | 3.84 | 3.84 | 3.87 |
| | | 3.87 | 3.86 | 3.86 |

The stability studies demonstrated that the amount of degradation product, pH, dissolution and NDMA content were found to be within specification.

Further stability study for composition of Example 3.D of batches was carried out for 40 °C/75% RH at 0, 3 and 6 months accelerated conditions.

| | | CONTENT OF | | |
|---|---|---|---|---|
| | | N-NITROSODIMETHYLAMINE | | |
| | | (NDMA) (ppm) by GC/MS | | |
| Batch | Stability condition | 0 | 3 months | 6 months |
| 1 | 40 +/− 2 °C/75 +/− | Not | 0.027 | Below |
| | 5% RH, Upright, | detected | | LOD |
| | 40 +/− 2 °C/75 +/− | Not | 0.022 | Below |
| | 5% RH, Horizontal, | detected | | LOD |
| 2 | 40 +/− 2 °C/75 +/− | Not | 0.017 | Below |
| | 5% RH, Upright, | detected | | LOD |
| | 40 +/− 2 °C/75 +/− | Not | 0.011 | Below |
| | 5% RH, Horizontal, | detected | | LOD |
| 3 | 40 +/− 2 °C/75 +/− | Not | Below | Below |
| | 5% RH, Upright, | detected | LOD | LOD |
| | 40 +/− 2 °C/75 +/− | Not | Below | Below |
| | 5% RH, Horizontal, | detected | LOD | LOD |

Further stability study of batches was carried out for 40 °C/75% RH at 6 months. Dissolution, impurity and pH data are provided in the table below. The stability products were placed into an inverted (or horizontal) position and an upright (or vertical) position at 40 °C/75% RH.

| Parameter | Specification | Batch 1 | Batch 2 | Batch 3 |
|---|---|---|---|---|
| Dissolution (Mean) | % Release in 5 | 86 | 86 | 90 |
| | hr = NLT 75% | 86 | 87 | 89 |
| Total Degradation | NMT 0.75% w/w | 0.05 | 0.06 | 0.07 |
| product | | 0.05 | 0.06 | Below limit |
| | | | | of disregard |
| pH | NMT 8.0 | 3.97 | 3.96 | 3.92 |
| | | 3.97 | 3.96 | 3.91 |

When the compositions of the current invention were subjected to stability studies, they maintained an NDMA impurity level within the allowable limits. This seems to indicate that the compositions remained stable throughout the entire stability period.

1. A two-part pharmaceutical composition of an amine drug or a pharmaceutically acceptable salt thereof and excipients, wherein the composition comprises:
a first part comprising:
a drug layered core, wherein the drug layer is coated on an inert core;
a water insoluble release-controlling polymer coated on the drug layered core;
a drug coating over the release control polymer coating, and
a second part comprising a vehicle devoid of drug;
wherein:
(i) the first part of the composition comprises a sulfite salt of alkali metal in close association with the drug layered core and in coating over the release control polymer; and/or
(ii) the second part of the composition comprises a buffering agent.

2. The two-part pharmaceutical composition as claimed in claim 1, wherein the ratio of the sulfite salt of alkali metal to drug in the composition is present in a ratio of 2.5:80 in the drug layered core and 2.5:20 in the drug coating.

3. The two-part pharmaceutical composition as claimed in claim 1, wherein the amine drug is selected from metformin, valsartan, losartan and/or ranitidine.

4. The two-part pharmaceutical composition as claimed in claim 1, wherein the release-controlling polymer is selected from ethyl cellulose, hydroxypropyl methyl cellulose, methyl cellulose, hydroxypropyl cellulose, methacrylic acid copolymer, polyethylene derivatives, gums, triglycerides, waxes and fatty acids derivatives or combination thereof.

5. The two-part pharmaceutical composition as claimed in claim 1, wherein the vehicle comprises a combination of osmogen and suspending agents.

6. The two-part pharmaceutical composition as claimed in claim 5, wherein the vehicle comprises one or more suspending agents, and the ratio of osmogen to suspending agent in the composition is 0.5:1 to 1:10.

7. The two-part pharmaceutical composition as claimed in claim 1, wherein the sulfite salt of alkali metal is selected from sodium sulfite, sodium bisulfite, sodium metabisulfite, potassium sulfite, potassium bisulfite, potassium metabisulfite, calcium sulfite, calcium bisulfite, calcium metabisulfite or a combination thereof.

8. The two-part pharmaceutical composition as claimed in claim 1, wherein:
(i) the excipients in the composition have a dimethyl amine content below 100 ppm;
(ii) the amine drug or pharmaceutically acceptable salt thereof is a low dimethyl amine containing drug comprising a DMA content below 300 ppm;
(iii) the dimethyl amine content of the amine drug or pharmaceutically acceptable salt thereof is less than 80 ppm;
(iv) the first part of the composition is a stable extended-release reconstitution powder for suspension composition comprising multiple coated cores of the active amine drug;
(v) the composition is characterized by having a nitrosamine drug substance related impurity below 0.048 ppm;
(vi) the composition has levels of NDMA of below 96 ng per Maximum Daily Dosage; or
(vii) any combination of (i)-(vi).

9-13. (canceled)

14. The two-part pharmaceutical composition as claimed in claim 1, wherein the buffering agent is selected from acetate buffer, citrate buffer and phosphate buffer.

15. The two-part pharmaceutical composition as claimed in claim 14, wherein the buffering agent maintains the pH between 2 and 10.

16. A pharmaceutical formulation comprising metformin as an amine-containing pharmaceutically active agent wherein said formulation exhibits levels of NDMA of below 0.048 ppm after storage for 7 days or longer.

17. A process for preparation of a stable pharmaceutical composition of an amine drug, which is characterised by having a nitrosamine drug substance related impurity below 0.048 ppm, said process comprising:
a) mixing a first fraction of low dimethyl amine active amine drug with a sulfite salt of an alkali metal and pharmaceutically acceptable excipients and coating the drug mixture on an inert core;
b) coating the core with a release-controlling polymer to obtain a coated drug core;
c) mixing a second fraction of low dimethyl amine active drug with a sulfite salt of an alkali metal and then coating the second fraction on the coated drug core;
d) coating the step c) resulting coated core with a top coating followed by lubrication;
e) separately preparing an aqueous vehicle by mixing osmogen in one or more suspending agents, and adding a buffering agent and a preservative to the mixture; and
f) packing separately the lubricated coated cores and vehicle.

18. A packaged pharmaceutical formulation comprising the two-part pharmaceutical composition of claim 1 in a packaging, wherein said packaging comprises an oxygen absorbent and/or is breathable or vented to allow diffusion of N-Nitrosamine out of the packaging.

19. The packaged pharmaceutical formulation as claimed in claim 18, wherein said packaging is breathable or vented to remove N-Nitrosamine formed during storage.

20. The packaged pharmaceutical formulation as claimed in claim 19, wherein said packaging is a vented blister pack.

21. The packaged pharmaceutical formulation as claimed in claim 20, wherein said packaging is a pill bottle containing an oxygen absorbent.

22. The packaged pharmaceutical formulation as claimed in claim 21, wherein said oxygen absorbent is silica.

23. The packaged pharmaceutical formulation as claimed in claim 18, wherein said N-Nitrosamine is NDMA.

24. The process as claimed in claim 17, wherein said amine-containing pharmaceutically active agent is metformin and the levels of NDMA after 14 days storage at 60 °C is 0.048 ppm or less.

25-37. (canceled)

38. A method of treating diabetes in a subject in need thereof, comprising administering the two-part pharmaceutical composition as claimed in claim 1 to the subject, wherein the amine drug is metformin.
